(19) United States
(12) Patent Application Publication
(10) Pub. No.: US 2003/0050307 A1
(43) Pub. Date: Mar. 13, 2003

(54) Novel indole derivatives

(75) Inventors: Thomas Ruhland, Valby (DK); Christian Krog-Jensen, Copenhagen (DK); Mario Rottlander, Valby (DK); Gitte Mikkelsen, Ballerup (DK); Kim Andersen, Virum (DK); Ejner Knud Moltzen, Gentofte (DK)

Correspondence Address: DARBY & DARBY P.C., 805 Third Avenue, New York, NY, 10022, US

(73) Assignee: H. Lundbeck A/S, Valby-Copenhagen (DK)

(21) Appl. No.: 10/183,961

(22) Filed: Jun. 25, 2002

Related U.S. Application Data
(63) Continuation of application No. PCT/DK00/00742, filed on Dec. 29, 2000.

(30) Foreign Application Priority Data
Dec. 30, 1999 (DK) PA 1999 01889

(51) Int. Cl. A61K 31/55; A61K 31/551; A61K 31/496; A61K 31/454; C 07D 4 3/02

(52) U.S. Cl. 514/218; 514/254.09; 540/575; 544/373; 546/201; 514/323

(57) ABSTRACT

The invention provide compounds of the formula

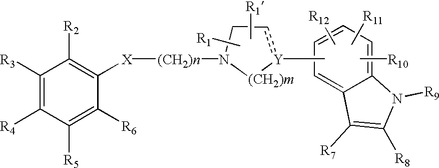

wherein

X represents O or S;

n is 2, 3, 4, 5, 6, 7, 8, 9 or 10;

m is 2 or 3;

Y represents N, C or CH;

and the dotted line represents an optional bond;

R¹, R^{1′}, R², R³, R⁴, R⁵, R⁶, R⁷, R⁸, R⁹, R¹⁰, R¹¹ and R¹² are as defined in the description.

The compounds are ligands of the 5-HT₁ₐ-receptor.

This application is a continuation of International Application No. PCT/DK00/00742, filed Dec. 29, 2000. The disclosure of the prior application is hereby incorporated by reference herein, in its entirety.

The present invention relates to novel indole derivatives potently binding to the 5-HT_{1A}-receptor, pharmaceutical compositions containing these compounds and the use thereof for the treatment of certain psychiatric and neurological disorders. Many of the compounds of to the invention are also potent serotonine reuptake inhibitors and/or D₄-ligands and are thus considered to be particularly useful for the treatment of depression and psychosis.

### BACKGROUND OF THE INVENTION

Clinical and pharmacological studies have shown that 5-HT_{1A}-agonists and partial agonists are useful in the treatment of a range of affective disorders such as generalised anxiety disorder, panic disorder, obsessive compulsive disorder, depression and aggression.

It has also been reported that 5-HT_{1A}-ligands may be useful in the treatment of ischemia.

An overview of 5-HT_{1A}-antagonists and proposed potential therapeutic

targets for these antagonists based upon preclinical and clinical data are presented by Schechter et al. Serotonin 1997, Vol.2, Issue 7. It is stated that 5-HT_{1A}-antagonists may be useful in the treatment of schizophrenia, senile dementia, dementia associated with Alzheimer's disease, and in combination with SSRI antidepressants also to be useful in the treatment of depression.

5-HT reuptake inhibitors are well-known antidepressant drugs and useful for the treatment of panic disorders and social phobia.

The effect of combined administration of a compound that inhibits serotonin reuptake and a 5-HT_{1A} receptor antagonist has been evaluated in several studies (Innis, R. B. et al. Eur. J. Pharmacol. 1987, 143, p 195-204 and Gartside, S. E., Br. J. Pharmacol. 1995, 115, p 1064-1070, Blier, P. et al. Trends Pharmacol. Sci. 1994, 15, 220). In these studies it was found that combined 5-HT_{1A}-receptor antagonists and serotonin reuptake inhibitors would produce a more rapid onset of therapeutic action.

Dopamine D₄-receptors belong to the family of dopamine D₂-like receptors, which are considered to be responsible for the antipsychotic effects of neuroleptics. Dopamine D₄-receptors are primarily located in areas of the brain other than striatum, suggesting that dopamine D₄-receptor ligands have antipsychotic effect and are devoid of extrapyramidal activity.

Accordingly, dopamine D₄ receptor ligands are potential drugs for the treatment of psychosis and positive symptoms of schizophrenia, and compounds with combined effects at dopamine D₄- and serotonergic receptors may have the further benefit of improved effect on negative symptoms of schizophrenia, such as anxiety and depression, alcohol abuse, impulse control disorders, aggression, side effects induced by conventional antipsychotic agents, ischemic disease states, migraine, senile dementia and cardiovascular disorders and in the improvement of sleep.

Dopamine D₃-receptors also belong to the family of dopamine D₂-like receptors. D₃-antagonistic properties of an antipsychotic drug could reduce the negative symptoms and cognitive deficits and result in an improved side effect profile with respect to EPS and hormonal changes.

Accordingly, agents acting on the 5-HT_{1A}-receptor, both agonists and antagonists, are believed to be of potential use in the therapy of psychiatric and neurological disorders and thus being highly desired. Furthermore, antagonists at the same time having potent serotonin reuptake inhibition activity and/or D₄ and/or D₃ activity may be particularly useful for the treatment of various psychiatric and neurological diseases.

Previously closely related structures have been reported:

WO 9955672 discloses a general formula of which indole derivatives are included having 5-HT_{1A} receptor and D₂ receptor affinity.

EP 900792 discloses a general formula of which indole derivatives are embraced as 5-HT_{1A}-and 5-HT_{1D} as well as D₂-receptor ligands.

It has now been found that a class of indole derivatives is particularly useful as 5-HT_{1A}-ligands.

Furthermore, it has been found that many of these compounds have other highly beneficial properties as e.g. potent serotonin reuptake inhibition activity and/or affinity for the D₄-receptor.

### SUMMARY OF THE INVENTION

The invention comprises the following:

A compound represented by the general formula I

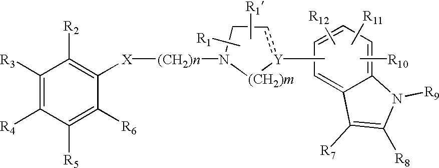

wherein

X represents O or S;

n is 2, 3, 4, 5, 6, 7, 8, 9 or 10;

m is 2 or 3;

Y represents N, C or CH;

and the dotted line represents an optional bond;

R¹ and R^{1′} independently represent hydrogen, or C¹⁻⁶-alkyl;

R⁷, R⁸, R¹⁰, R¹¹ and R¹² are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₆₋-alkyl, C₁₋₆-alkoxy, C₁₋₆-alkylsulfanyl, hydroxy, formyl, acyl, amino, C₁₋₆-alkylamino, di(C₁₋₆-alkyl)amino, acylamino, C₁₋₆-alkoxycarbonylamino, aminocarbonylamino, C₁₋₆-alkylaminocarbonylamino and di(C₁₋₆-alkyl)aminocarbonylamino;

R⁹ represents hydrogen, C, ₆-alkyl or acyl;

R², R³, R⁴, R⁵ and R⁶ independently represent hydrogen, halogen, cyano, nitro, C₁₋₆-alkyl, C₁₋₆-alkoxy, C₁₋₆-alkylsulfanyl, C₁₋₆ alkylsulfonyl, hydroxy, hydroxy-C₁₋₆-alkyl, C₁₋₆-alkoxycarbonyl, acyl, C₃₋₈-cycloalkyl, C₃₋₈-cycloalkyl-C₁₋₆-alkyl, trifluoromethyl, trifluoromethoxy, NH₂, NR¹³R¹⁴ wherein R¹³ and R¹⁴ independently represent hydrogen, C₁₋₆-alkyl, C₃₋₈-cycloalkyl, or phenyl; or R¹³ and R¹⁴ together with the nitrogen to which they are attached form a 5- or 6-membered carbocyclic ring optionally containing one further heteroatom;

its enantiomers, and a pharmaceutically acceptable acid addition salt thereof.

The invention provides a pharmaceutical composition comprising at least one compound of Formula I as defined above or a pharmaceutically acceptable acid addition salt thereof or prodrug thereof in a therapeutically effective amount and in combination with one or more pharmaceutically acceptable carriers or diluents.

The present invention provides the use of a compound of Formula I as defined above or an acid addition salt or prodrug thereof for the manufacture of a pharmaceutical preparation for the treatment of diseases and disorders responsive to ligands of the 5-HT₁ₐ-receptor potentially in combination with serotonine reuptake and/or ligands at the dopamine D₄ receptor.

The invention further provides a method for the treatment of diseases and disorders in humans responsive to ligands of the 5-HT₁ₐ-receptor potentially in combination with serotonine reuptake and/or ligands at the dopamine D₄-receptor, comprising administering an effective amount of a compound of Formula I.

The diseases and disorders to be treated by administration of compounds of the present invention are: affective disorders such as generalised anxiety disorder, panic disorder, obsessive compulsive disorder, depression, social phobia, eating disorders, and aggression, and neurological disorders such as psychosis.

### DETAILED DESCRIPTION OF THE INVENTION

A preferred embodiment of the invention is a compound of formula I as above, wherein

X represents O or S;

n is 2, 3, 4 or 5

m is 2 or 3;

Y represents N or CH;

R¹ and R^{1′} are both hydrogen;

one or two of R⁷, R⁸, R¹⁰, R¹¹ and R¹² independently represent hydrogen, halogen, CF₃, CN or C₁₋₆-alkyl; and the remaining of R⁷, R⁸, R¹⁰, R¹¹ and R¹² represent hydrogen;

R⁹ represents hydrogen;

R², R³, R⁴, R⁵ and R⁶ independently represent

hydrogen, halogen, C₁₋₆-alkyl, C₃₋₈-cycloalkyl, C₁₋₆-alkoxy, hydroxy, nitro, CN, CF₃, OCF₃, acyl; NH₂, NR¹³R¹⁴ wherein R¹³ and R¹⁴ independently represent hydrogen, C₁₋₆-alkyl, C₃₋₈-cycloalkyl, or phenyl; or R¹³ and R¹⁴ together with the nitrogen form a piperidine, morpholine, piperazine or pyrrolidine; its enantiomers, and a pharmaceutically acceptable acid addition salt thereof.

In a further embodiment of the invention, the compound of formula I as described above wherein R¹ and R^{1′} are hydrogen.

In a further embodiment of the invention, the compound of formula I as described above wherein m is 2.

In a further embodiment of the invention, the compound of formula I as described above wherein n is 2, 3 or 4;

In a further embodiment of the invention, the compound of formula I as described above wherein Y is N;

In a further embodiment of the invention, the compound of formula I as described above wherein the indole is attached to the group Y in position 4.

A further embodiment of the invention is a compound of formula I as described above wherein at least one of R², R³, R⁴, R⁵ and R⁶ is representing halogen;

In a further embodiment of the invention, the compound of formula I as described above wherein at least two of R², R³, R⁴, R⁵ and R⁶ represent halogen;

In a further embodiment of the invention, the compound of formula I as described above wherein at least three of R², R³, R⁴, R⁵ and R⁶ represent halogen;

In a further embodiment of the invention, the compound of formula I as described above wherein R² and/or R⁶ are not hydrogen.

In a preferred embodiment of the invention, the compound of formula I as described above are

4-{4-[3-(2-Chloro-phenoxy)-propyl]-piperazin-1-yl}-1H-indole

4-{4-[3-(2-Chloro-phenylsulfanyl)-propyl]-piperazin-1-yl}-1H-indole

4-{4-[3-(2-Bromo-phenylsulfanyl)-propyl]-piperazin-1-yl}-1H-indole

4-{4-[3-(2-Bromo-phenoxy)-propyl]-piperazin-1-yl}-1H-indole

4-{4-[4-(2-Bromo-4-fluoro-phenoxy)-butyl]-piperazin-1-yl}-1H-indole

4-{4-[4-(2-Chloro-6-methyl-phenylsulfanyl)-butyl]-piperazin-1-yl}-1H-indole

4-{4-[2-(2-Chloro-4-fluoro-phenylsulfanyl)-ethyl]-piperazin-1-yl}-1H-indole

4-{4-[2-(2,6-Dichloro-phenylsulfanyl)-ethyl]-piperazin-1-yl}-1H-indole

4-{4-[2-(3,4-Dichloro-phenylsulfanyl)-ethyl]-piperazin-1-yl}-1H-indole

4-{4-[2-(4-Fluoro-phenylsulfanyl)-ethyl]-piperazin-1-yl}-1H-indole

4-{4-[3-(2-Chloro-4-fluoro-phenylsulfanyl)-propyl]-piperazin-1-yl}-1H-indole

4-{4-[4-(2-Bromo-4-fluoro-phenoxy)-butyl]-piperazin-1-yl}-1H-indole

4-{4-[3-(2,4-Difluoro-phenoxy)-propyl]-piperazin-1-yl}-1H-indole

4-{4-[4-(2,6-Dichloro-phenylsulfanyl)-butyl]-piperazin-1-yl}-1H-indole

4-{4-[3-(2-Chloro-4-fluoro-phenoxy)-propyl]-piperazin-1-yl}-1H-indole

4-{4-[4-(2-Chloro-6-methyl-phenylsulfanyl)-butyl]-piperazin-1-yl}-1H-indole

4-{4-[4-(2,6-Dichloro-4-fluoro-phenoxy)-butyl]-piperazin-1-yl}-1H-indole

4-{4-[3-(2-Bromo-4,6-difluoro-phenoxy)-propyl]-piperazin-1-yl}-1H-indole

4-{4-[3-(2,6-Dichloro-4-fluoro-phenoxy)-propyl]-piperazin-1-yl}-1H-indole

4-{4-[4-(4-Bromo-2,6-difluoro-phenoxy)-butyl]-piperazin-1-yl}-1H-indole

4-{4-[4-(2,6-Dibromo-4-fluoro-phenoxy)-butyl]-piperazin-1-yl}-1H-indole

4-{4-[3-(2,4,6-Tribromo-phenoxy)-propyl]-piperazin-1-yl}-1H-indole

4-{4-[3-(4-Bromo-2,6-difluoro-phenoxy)-propyl]-piperazin-1-yl}-1H-indole

1-(3,5-Difluoro-4-{3-[4-(1H-indol-4-yl)-piperazin-1-yl]-propoxy}-phenyl)-propan-1-one

3,5-Dibromo-4-{3-[4-(1H-indol-4-yl)-piperazin-1-yl]-propoxy}-benzonitrile

4-{4-[2-(2-Bromo-4,6-difluoro-phenoxy)-ethyl]-piperazin-1-yl}-1H-indole

4-{4-[3-(2,6-Dichloro-phenylsulfanyl)-propyl]-piperazin-1-yl}-1H-indole

4-{4-[2-(2,6-Dimethyl-phenoxy)-ethyl]-piperazin-1-yl}-1H-indole

4-{4-[4-(2,6-Dimethyl-phenylsulfanyl)-butyl]-piperazin-1-yl}-1H-indole

4-{4-[2-(2,4-Dimethyl-phenylsulfanyl)-ethyl]-piperazin-1-yl}-1H-indole

4-{4-[2-(2,3-Dichloro-phenylsulfanyl)-ethyl]-piperazin-1-yl}-1H-indole

4-{4-[2-(2-Allyl-6-chloro-phenoxy)-ethyl]-piperazin-1-yl}-1H-indole

4-{4-[3-(2-Trifluoromethyl-phenylsulfanyl)-propyl]-piperazin-1-yl}-1H-indole

4-{4-[3-(3,4-Dichloro-phenylsulfanyl)-propyl]-piperazin-1-yl}-1H-indole

4-{4-[4-(2,4-Dimethyl-phenoxy)-butyl]-piperazin-1-yl}-1H-indole

4-{4-[4-(2-Ethyl-phenoxy)-butyl]-piperazin-1-yl}-1H-indole

4-[4-(4-Phenylsulfanyl-butyl)-piperazin-1-yl]-1H-indole

4-{4-[4-(2-Chloro-5-methyl-phenoxy)-butyl]-piperazin-1-yl}-1H-indole

4-{4-[2-(2,5-Dichloro-phenylsulfanyl)-ethyl]-piperazin-1-yl}-1H-indole

4-{4-[2-(3-Chloro-phenylsulfanyl)-ethyl]-piperazin-1-yl}-1H-indole

4-{4-[2-(2-Chloro-phenylsulfanyl)-ethyl]-piperazin-1-yl}-1H-indole

4-{4-[3-(3-Chloro-phenylsulfanyl)-propyl]-piperazin-1-yl}-1H-indole

3-Chloro-4-{4-[4-(1H-indol-4-yl)-piperazin-1-yl]-butoxy}-benzonitrile

4-{4-[4-(3-Chloro-phenylsulfanyl)-butyl]-piperazin-1-yl]-1H-indole

4-{4-[4-(2-Chloro-phenylsulfanyl)-butyl]-piperazin-1-yl}-1H-indole

4-{4-[3-(3,4-Dimethyl-phenylsulfanyl)-propyl]-piperazin-1-yl}-1H-indole

3-{4-[4-(1H-Indol-4-yl)-piperazin-1-yl]-butoxy}-benzonitrile

4-{4-[4-(2,5-Dichloro-phenoxy)-butyl]-piperazin-1-yl}-1H-indole

4-{4-[4-(3,4-Dimethoxy-phenylsulfanyl)-butyl]-piperazin-1-yl}-1H-indole

4-{4-[3-(4-Trifluoromethyl-phenylsulfanyl)-propyl]-piperazin-1-yl}-1H-indole

4-{4-[3-(4-Trifluoromethoxy-phenylsulfanyl)-propyl]-piperazin-1-yl}-1H-indole

4-{4-[3-(3T-Bromo-phenylsulfanyl)-propyl]-piperazin-1-yl}-1H-indole

4-{4-[3-(2-Isopropyl-phenylsulfanyl)-propyl]-piperazin-1-yl}-1H-indole

4-{4-[4-(2-Methoxy-phenoxy)-butyl]-piperazin-1-yl}-1H-indole or

4-{4-[4-(2-Isopropyl-phenylsulfanyl)-butyl]-piperazin-1-yl}-1H-indole

or a pharmaceutical acceptable salt thereof.

Definition of Substituents etc.

The term C₁₋₆ alkyl refers to a branched or linear alkyl group having from one to six carbon atoms inclusive, including but not limited to methyl, ethyl, 1-propyl, 2-propyl, 1-butyl, 2-butyl, 2-methyl-2-propyl and 2-methyl-1-propyl.

Similarly, C₂₋₆ alkenyl and C₂₋₆ alkynyl, respectively, designate such groups having from two to six carbon atoms, inclusive and the groups are having at least one double bond or triple bond, respectively;

The terms C₁₋₆-alkoxy, C₁₋₆ alkylsulfanyl, C₁₋₆ alkylsulfonyl, C₁₋₆ alkylamino, C₁₋₆ alkylcarbonyl, hydroxy-C₁₋₆-alkyl etc. designate such groups in which the C₁₋₆ alkyl is as defined above.

The term C₃₋₈ cycloalkyl designates a monocyclic or bicyclic carbocycle having three to eight C-atoms, including but not limited to cyclopropyl, cyclobutyl, cyclopentyl, cyclohexyl, etc.

The term aryl refers to a carbocyclic aromatic group, such as phenyl, naphthyl, in particular phenyl. As used herein, aryl may be substituted one or more times with halogen, nitro, cyano, trifluoromethyl, C₁₋₆-alkyl, hydroxy and C₁₋₆-alkoxy.

Halogen means fluoro, chloro, bromo or iodo.

As used herein, the term acyl refers to formyl, C₁₋₆-alkylcarbonyl, arylcarbonyl, aryl-C₁₋₆-alkylcarbonyl wherein the aryl is as defined above; C₃₋₈-cycloalkylcarbonyl, or a C₃₋₈-cycloalkyl-C₁₋₆alkyl-carbonyl group.

The terms amino, C₁₋₆-alkylamino and C₂₋₁₂-dialkylamino means respectively NH₂, NH(C₁₋₆alkyl) wherein alkyl is as defined above; and N(C₁₋₆-alkyl)₂ wherein alkyl is as defined above.

The term acylamino means —CO-amino wherein amino is defined as above.

The term aminocarbonyl means a group of the formula —NHCOH, —NHCO—C₁₋₆-alkyl, —NHCO-aryl, —NHCO-C₃₋₈-cycloalkyl, —NHCO—C₃₋₈-cycloalkyl-C₁₋₆alkyl wherein the alkyl, cycloalkyl and aryl are as defined above.

The terms aminocarbonylamino, C₁₋₆-alkylaminocarbonylamino and di(C₁₋₆-alkyl)aminocarbonylamino means a group of the formula NHCONH₂, —NHCONHC₁₋₆-alkyl, NHCON(di-C₁₋₆-alkyl).

The acid addition salts of the invention are preferably pharmaceutically acceptable salts of the compounds of the invention formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids.

Further, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

Some of the compounds of the present invention contain chiral centres and such compounds exist in the form of isomers (i.e. enantiomers). The invention includes all such isomers and any mixtures thereof including racemic mixtures.

Racemic forms can be resolved into the optical antipodes by known methods, for example, by separation of diastereomeric salts thereof with an optically active acid, and liberating the optically active amine compound by treatment with a base. Another method for resolving racemates into the optical antipodes is based upon chromatography on an optically active matrix. Racemic compounds of the present invention can also be resolved into their optical antipodes, e.g., by fractional crystallization of d- or 1-(tartrates, mandelates or camphorsulphonate) salts for example. The compounds of the present invention may also be resolved by the formation of diastereomeric derivatives.

Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981).

Optically active compounds can also be prepared from optically active starting materials.

Finally, formula (I) includes any tautomeric forms of the compounds of the invention.

The compounds of the invention can be prepared by one of the following methods comprising:

a) reducing the carbonyl groups of a compound of formula

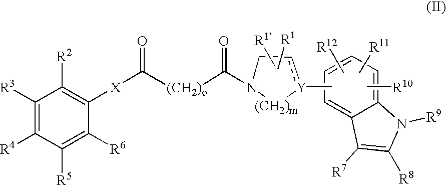

wherein o=0-8, R¹—R¹², X, Y, m, and the dotted line are as defined above;

b) reducing the carbonyl group of a compound of formula

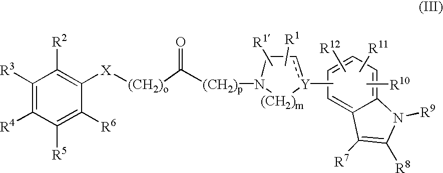

wherein o=0-9, p=0-4, and with the proviso that o+p is not greater than 9; R¹—R¹², X, Y, m, and the dotted line are as defined above;

c) alkylating an amine of formula

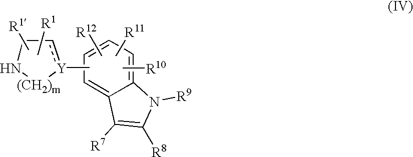

wherein R¹, R⁷—R¹²,Y, Ym, and the dotted line are as defined above with a reagent of formula

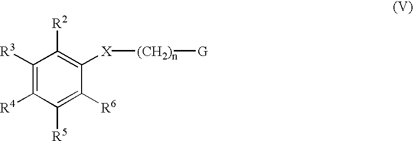

wherein G is a suitable leaving group such as halogen, mesylate or tosylate; and R²—R⁶, X and n are as defined above

d) reductive alkylation of an amine of formula

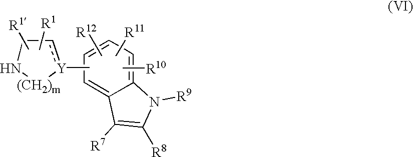

with a reagent of formula

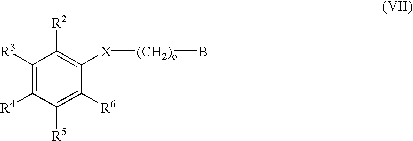

wherein R¹—R¹², Y, X, m and n and the dotted line are as defined above and B is either an aldehyde or a carboxylic acid derivative;

e) oxidation of 2,3-dihydroindoles of formula

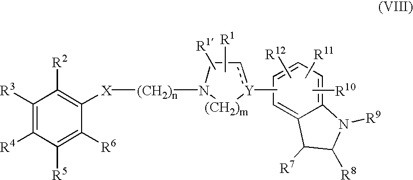

wherein R¹—R¹², Y, X, n and m and the dotted line are as defined above

f) reducing the double bond of unsaturated cyclic amines of formula

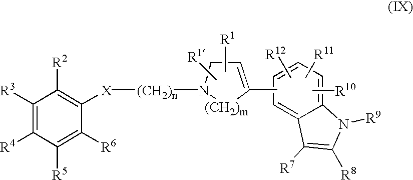

wherein R¹—R¹², X, n and m are as previously defined, in order to obtain the corresponding saturated derivatives;

g) reductive removal of one or more of the substituents R¹-R³ or R⁷—R¹² in a compound of general formula (I) in which one or more of these substituents are selected from chloro, bromo or iodo;

h) dialkylating an amine of formula

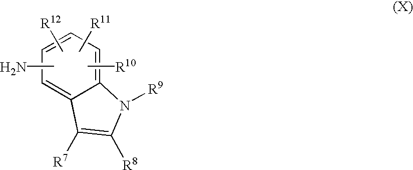

with a reagent of formula

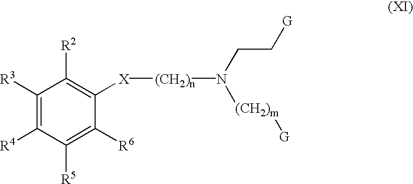

wherein R¹—R¹², Y, X, n and m are as defined above and G is a suitable leaving group such as halogen, mesylate or tosylate;

i) dialkylating an amine of formula

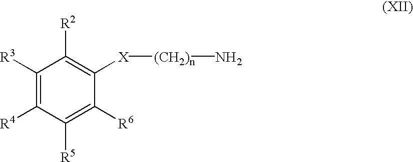

wherein R²—R⁶, X and n are as defined above, with a reagent of formula

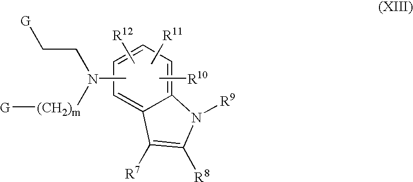

wherein R⁷—R¹² and m are as defined above and G is a suitable leaving group such as halogen, mesylate or tosylate; or

j) alkylating or acylating the indole nitrogen atom of compounds of formula

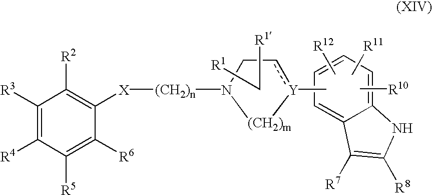

wherein R¹—R¹², Y, X, n and m, and the dotted line are as defined above; R⁹ is hydrogen with alkylating or acylating reagents of formula R⁹-G, wherein G suitably is a leaving group such as halogen, mesylate, or tosylate and R⁹ is as defined above but not hydrogen;

k) reduction of sulfones or sulfoxides of the formula

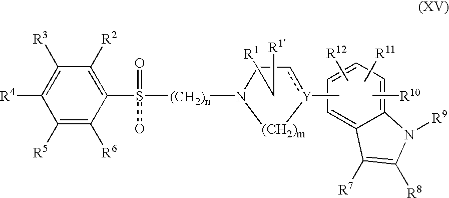

wherein R¹—R¹², Y, m and n are as defined above and the dotted lines are optional bonds;

m) alkylation of compounds of formula

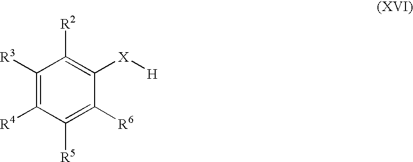

wherein R²—R⁶ and X are as defined above with a suitable derivatised compound including a leaving group to form a compound of the invention.

The compounds of formula (I) are isolated as the free base or in the form of a pharmaceutically acceptable salt thereof.

The reduction according to method a) and b) is preferably carried out in an inert organic solvent such as diethyl ether or tetrahydrofuran in the presence of lithium aluminium hydride at reflux temperature.

The alkylation according to method c) is conveniently performed in an inert organic solvent such as a suitable boiling alcohol or ketone, preferably in the presence of a base (potassium carbonate or triethylamine) at reflux temperature.

Arylpiperazine derivatives of formula (IV) are commercially available but can also be conveniently prepared from the corresponding arylamine according to the method described by Martin et al. J. Med. Chem. 1989, 32, 1052, or the method described by Kruse et al. Rec. Trav. Chim. Pays-Bas 1988, 107. The starting arylamines are either commercially available or are well-described in the literature.

Aryltetrahydropyridine derivatives of formula (IV) are known from literature, cf. U.S. Pat. No. 2,891,066; McElvain et al. J. Amer. Chem. Soc. 1959, 72, 3134. Conveniently, the corresponding arylbromide is lithiated with BuLi followed by addition of 1-benzyl-4-piperidone. Subsequent treatment with acid gives the N-benzyl-aryltetrahydropyridine. The benzyl group can be removed by catalytic hydrogenation or by treatment with e.g. ethyl chloroformate to give the corresponding ethyl carbamate followed by acidic or alkaline hydrolysis. The starting arylbromides are either commercially available or well-described in the literature.

Reagents of formula (V) are either commercially available or can be prepared by literature methods, e.g. from the corresponding carboxylic acid derivative by reduction to the 2-hydroxyethyl derivative and conversion of the hydroxy group to the group G by conventional methods, or from the corresponding dihalo alkyl or 1-halo alkohol.

The reductive alkylation according to method d) is performed by standard literature methods. The reaction can be performed in two steps, i.e. coupling of (IV) and the reagent of formula (VII) by standard methods via the carboxylic acid chloride or by use of coupling reagents such as e.g. dicyclohexylcarbodiimide followed by reduction of the resulting amide with lithium aluminium hydride. The reaction can also be performed by a standard one-pot procedure. Carboxylic acids or aldehydes of formula (VII) are either commercially available or described in the literature.

Oxidation of 2,3-dihydroindole according to method e) is conveniently performed by treatment with palladium on carbon in refluxing p-xylene or methanol (Aoki et al. J. Am. Chem. Soc. 1998,120, 3068-3073 and Bakke, J. Acta Chem Scand. 1974, B28, 134-135).

Reduction of the double bonds according to methods f) is most conveniently performed by hydrogenation in an alcohol in the presence of a noble metal catalyst, such as e.g. platinum or palladium.

The removal of halogen substituents according to method g) is conveniently performed by catalytic hydrogenation in an alcohol in the presence of a palladium catalyst or by treatment with ammonium formate in an alcohol at elevated temperatures in the presence of a palladium catalyst.

The dialkylation of amines according to methods h) and i) is most conveniently performed at elevated temperatures in an inert solvent such as e.g. chlorobenzene, toluene, N-methylpyrrolidone, dimethylformamide or acetonitrile. The reaction might be performed in the presence of base such as e.g. potassium carbonate or triethylamine. Starting materials for processes h) and i) are commercially available or can be prepared from commercially available materials using conventional methods.

The N-alkylation according to method j) is performed in an inert solvent such as e.g. an alcohol or ketone at elevated temperatures in the presence of base e.g. potassium carbonate or triethylamine at reflux temperature. Alternatively, a phase-transfer reagent can be used.

Reduction of sulfones and sulfoxides according to method k) can be performed using several commercially available reagents as titaniumtetrachloride and sodiumborohydride at room temperature (S. Kano et al. Synthesis 1980, 9, 695-697).

Alkylation of commercially available compounds corresponding to formula XVI using method m) is conveniently performed using a alkylating reagent with the appropriate leaving group (e.g. mesylate, halide) using a base (e.g. potassium carbonate or similar) in an polar aprotic solvent (e.g. methyl isobutylketone, dimethylformamide).

Halogen-, methyl- or methoxy substituted indoles used as described in the examples are commercially available.

Substituted 2-(1-indolyl)acetic acids used as described the examples are prepared from the corresponding substituted indole and ethyl bromoacetate by conventional methods.

Substituted 3-(2-bromoethyl)indoles used as described in the examples are prepared from the corresponding in 2-(1-indolyl)acetic acid ester by reduction to the alcohol with lithium aluminium hydride and subsequent treatment with tetrabromomethane/triphenylphosphine according to standard literature methods.

Arylpiperazines used as described in the examples are prepared from the corresponding arylamine according to the method described by Martin et al. J. Med. Chem. 1989, 32,1052, or the method described by Kruse et al. Rec. Trav. Chim. Pays-Bas 1988, 107, 303.

The following examples will illustrate the invention further. They are, however, not to be construed as limiting.

### EXAMPLES

Melting points were determined on a Büchi SMP-20 apparatus and are uncorrected. Analytical LC-MS data were obtained on a PE Sciex API 150EX instrument equipped with IonSpray source (method D) or heated nebulizer (APCI, methods A and B) and Shimadzu LC-8A/SLC-10A LC system. The LC conditions [30×4.6 mm YMC ODS-A with 3.5 Em particle size] were linear gradient elution with water/acetonitrile/trifluoroacetic acid (90:10:0.05) to water/acetonitrile/trifluoroacetic acid (10:90:0.03) in 4 min at 2 mL/min. Purity was determined by integration of the UV trace (254 nm). The retention times Rt are expressed in minutes.

Mass spectra were obtained by an alternating scan method to give molecular weight information. The molecular ion, MH+, was obtained at low orifice voltage (5-20V) and fragmentation at high orifice voltage (100V).

Preparative LC-MS-separation was performed on the same instrument. The LC conditions (50×20 mm YMC ODS-A with 5 μm particle size) were linear gradient elution with water/acetonitrile/trifluoroacetic acid (80:20:0.05) to water/acetonitrile/trifluoroacetic acid (10:90:0.03) in 7 min at 22.7 mL/min. Fraction collection was performed by split-flow MS detection.

¹H NMR spectra were recorded at 500.13 MHz on a Bruker Avance DRX500 instrument or at 250.13 MHz on a Bruker AC₂₅₀ instrument. Deuterated chloroform (99.8%D) or dimethyl sulfoxide (99.9%D) were used as solvents. TMS was used as internal reference standard. Chemical shift values are expressed in ppm-values. The following abbreviations are used for multiplicity of NMR signals: s=singlet, ddoublet, t=triplet, q=quartet, qui=quintet, h=heptet, dd=double doublet, dt=double triplet, dq=double quartet, tt—triplet of triplets, m=multiplet, b=broad singlet. NMR signals corresponding to acidic protons are generally omitted. Content of water in crystalline compounds was determined by Karl Fischer titration. Standard workup procedures refer to extraction with the indicated organic solvent from proper aqueous solutions, drying of combined organic extracts (anhydrous MgSO₄ or Na₂SO₄), filtering and evaporation of the solvent in vacuo. For column chromatography, silica gel of type Kieselgel 60, 230-400 mesh ASTM was used. For ion-exchange chromatography, SCX, 1 g, Varian Mega Bond Elut®, Chrompack cat. no. 220776 was used. Prior use the SCX-columns were pre-conditioned with 10% solution of acetic acid in methanol (3 mL).

### Example 1

1a. 4-{4-[3-(2-Chloro-phenoxy)-propyl]-piperazin-1-yl}-1H-indole.

A solution of 2-chlorophenol (5 g) in tetrahydrofuran (25 mL) was added dropwise to a slurry of sodium hydride (47 mmol) in tetrahydrofuran (50 mL) at room temperature. The mixture was stirred for 30 min. The reaction mixture was then warmed to reflux whereafter 2-bromo-1-propanol (3.5 mL) in tetrahydrofuran (25 mL) was added over 5 min. The mixture was refluxed over night, one more equivalent of 3-bromo-1-propanol was added and the mixture was refluxed for 12 h more. The mixture was cooled, brine and ethyl acetate added, and washed using standard procedure. The combined organic phases were dried and evaporated. The crude product, 3-(2-chlorophenoxy)-1-propanol, was dissolved in acetonitrile (500 mL) and carbontetrabromide (38.7 g) was added. To the cooled (0° C.) mixture, triphenylphosphine (25.5 g) was added portionwise over 30 min. The reaction was allowed to react at room temperature for 3 h, then evaporated to give an oily product. The crude product was purified using silica gel flash chromatography (heptane: ethylacetate: triethylamine/70:15:5) to give 3-(2-chlorophenoxy)-1-propylbromide (10.7 g). A mixture of (1H-indole-4-yl)piperazine (0.77 g), potassium carbonate (1.6 g), potassium iodide (cat.) and 3-(2-chlorophenoxy)-1-propylbromide (1.0 g) in methyl isobutylketone/dimethylformamide (1/1, 100 mL) was heated to 120° C. When TLC indicated the reaction to be completed (24 h), the mixture was cooled, filtered and evaporated. The crude material was dissolved in ethyl acetate and washed using standard procedure, followed by drying, filtration and evaporation. The crude material was purified using silicagel flash chromatography (heptane: ethylacetate: triethylamine/55:43:2). The collected pure oil was dissolved in ethanol followed by addition of etheral hydrogen chloride. Filtration gave the title compound as pure crystalline material (0.3 g). Mp. 189-99° C. ¹H NMR (DMSO-d₆): 2.30 (m, 2H); 3.20-3.45 (m, 6H); 3.60-3.75 (m, 4H); 4.20 (t, 2H); 6.45 (m, 1H); 6.55 (d, 1H); 6.95-7.05 (m, 2H); 7.10-7.20 (m, 2H); 7.25-7.35 (m, 2H); 7.45 (d, 1H); 11.05 (b, 1H); 11.20 (s, 1H). MS: m/z: 370 (MH+), 199, 117. Anal. Calcd for C₂₁H₂₄ClN₃O: C, 54.72; H, 6.14; N, 9.12. Found C, 55.20; H, 6.48; N, 8.45.

### Example 2

2a, 4-{4-[3-(2-Chloro-phenylsulfanyl)-propyl]-piperazin-1-yl}-1H-indole, 0.75 oxalate.

A solution of 2-chlorothiophenol (5g) in dimethylformamide (50 mL) was added dropwise to a slurry of sodiumhydride (38 mmol) in dimethylformamide at room temperature, over 15 min. The mixture was stirred for 30 min. The reaction mixture was then added slowly (10 min) to a solution of 1,3-dibromopropane in dimethylformamide (25 mL) at room temperature. The final mixture was stirred for further 60 min. The reaction was quenched by addition of sufficient amounts of water to consume the excess of sodiumhydride, acidified using etheral hydrogen chloride followed by evaporation. The crude product was purified using silicagel flash chromatography, (heptane: ethylacetate: triethylamine/95:2.5:2.5) to give 3-(2-chlorophenylthio)-1-propylbromide (5.7 g).

A mixture of (1H-indole-4-yl)piperazine (1.1 g), potassium carbonate (2.3 g), potassium iodide (cat.) and 3-(2-chlorophenylthio)-1-propylbromide (1.5 g) in methyl isobutylketone/dimethylformamide (1/1, 100 mL) was heated to 120° C. When TLC indicated the reaction to be completed (24 h), the mixture was cooled, filtered and evaporated. The crude material was dissolved in ethyl acetate and washed using standard procedure, followed by drying, filtration and evaporation. The crude materials were purified using silicagel flash chromatography (heptane: ethylacetate: ethanol: triethylamine/85:5:25:5). The collected pure oil was dissolved in ethanol (150 mL) followed by addition of oxalic acid. Filtration gave the title compound as pure crystalline material (1.2 g). Mp. 182-83° C. ¹H NMR (DMSO-d₆): 1.95 (q, 2H); 2.75-3.00 (m, 6H); 3.10 (t, 2H); 3.15-3.25 (m, 4H); 6.40 (m, 1H); 6.45 (d, 1H); 6.95-7.05 (m, 2H); 7.15-7.25 (m, 2H); 7.35 (t, 1H); 7.40-7.50 (m, 2H); 11.05 (s, 1H). MS: m/z: 386 (MH+), 285, 157. Anal. Calcd for C₂₁H₂₄ClN₃S: C, 59.58; H, 5.68; N, 9.27. Found C, 59.28; H, 6.01; N, 9.33.

The following compounds were prepared analogously:

2b, 4-(4-[3-(2-Bromo-phenylsulfanyl)-propyl]-piperazin-1-yl}-1H-indole, Oxalate

Mp. 163-66° C. ¹H NMR (DMSO-d₆): 1.95 (q, 2H); 3.00 (t, 2H); 3.00-3.15 (m, 6H); 3.20-3.35 (m, 4H); 6.40 (m, 1H); 6.45 (d, 1H); 6.95-7.15 (m, 3H); 7.25 (m, 1H); 7.40 (m, 2H); 7.60 (d, 1H); 11.05 (s, 1H). MS: m/z: 430 (MH+), 229, 159. Anal. Calcd for C₂₁H₂₄BrN₃S: C, 53.07; H, 5.05; N, 8.08. Found C, 52.83; H, 5.34; N, 8.14.

2c, 4-{4-[3-(2-Bromo-phenoxy)-propyl]-piperazin-1-yl}-1H-indole, Hemioxalate.

Mp. 206-8° C. ¹H NMR (DMSO-d₆): 2.05 (q, 2H); 2.85-3.05 (m, 6H); 3.15-3.30 (m, 4H); 4.15 (t, 2H); 6.40 (m, 1H); 6.45 (d, 1H); 6.85-7.10 (m, 3H); 7.15 (d, 1H); 7.25 (m, 1H); 7.35 (m, 1H); 7.55 (d, 1H); 11.05 (s, 1H). MS: m/z: 416, 414 (MH+), 258, 199, 159. Anal. Calcd for C₂₁H₂₄BrN₃O: C, 57.51; H, 5.50; N, 9.15. Found C, 57.53; H, 5.59; N, 8.98.

2d, 4-{4-[4-(2-Bromo-4-fluoro-phenoxy)-butyl]-piperazin-1-yl}-1H-indole, Oxalate.

Mp. 218-20° C. ¹H-NMR (DMSO-d₆): 1.75-1.95 (m, 4H); 3.15-3.25 (t, 2H); 3.20-3.40 (m, 8H); 4.05-4.15 (t, 2H); 6.40-6.45 (s, 1H); 6.45-6.50 (d, 1H); 6.95-7.00 (t, 1H); 7.05-7.10 (d, 1H); 7.10-7.25 (m, 2H); 7.25-7.30 (m, 1H); 7.50-7.60 (dd, 1H). MS m/z: 446 (MH+), 371, 247, 149. Anal. Calcd for C₂₂H₂₅BrFN₃O: C, 53.73; H, 5.08; N, 7.84. Found C, 54.77; H, 5.38; N, 7.60.

2e, 4-{4-[4-(2-Chloro-6-methyl-phenylsulfanyl)-butyl]-piperazin-1-yl}-1H-indole, Oxalate.

Mp. 199-210° C. ¹H-NMR (DMSO-d₆): 1.45-1.60 (m, 2H);1.70-1.85 (m, 2H); 2.55 (s, 3H); 2.80-2.90 (t, 2H); 2.95-3.05 (t, 2H); 3.15-3.40 (m, 8H); 6.40-6.45 (s, 1H); 6.45-6.50 (d, 1H); 6.95-7.05 (t, 1H); 7.05-7.10 (d, 1H); 7.25-7.35 (m, 3H); 7.35-7.45 (dd, 1H); 11.05-11.15 (s, 1H). MS m/z: 414 (MH+), 256, 213, 149.

Anal. Calcd for C₂₂H₂₅ClN₃S: C, 59.56; H, 6.01; N, 8.34. Found C, 60.10; H, 6.15; N, 8.20.

### Example 3

3a, 4-{4-[2-(2-Chloro-4-fluoro-phenylsulanyl)-ethyl]-piperazin-1-yl}-1H-indole, 1.25 Oxalate

A solution of chloroacetyl chloride (1.86 g) in dry tetrahydrofuran (5 mL) was added dropwise over 10 min to a mixture of (1H-indole-4-yl)piperazine (2.50 g) and triethylamine (3.8 g) in dry tetrahydrofuran at room temperature. The reaction was quenched with water after 40 min and washed using standard procedure (ethyl acetate). Drying and evaporation gave 3.5 g of the chloroacetylated derivative. This crude product was directly used in the subsequent step. 2-chloro-4-fluorothiophenol (1.1 g) was dissolved in tetrahydrofuran (40 mL) and potassium tert-butoxide (0.84 g) was added followed by stirring for 10 min. This mixture was treated dropwise with a solution of the chloroacetylated derivative (1.70 g), prepared above, in tetrahydrofuran (20 mL). The reaction was allowed to proceed at room temperature for 1 h and then 20 min at reflux, whereafter is was cooled and evaporated. The crude mixture was washed using standard procedure (ethyl acetate) and evaporated to give, after purification by silicagel flash chromatography (heptane: 30-50% ethylacetate), the pure alkylated product (2.00 g), 1-[2-chloro-4-fluorophenylthiomethylcarbonyl]-4-[1H-indol-4-yl]piperazine.

Aluminium trichloride (0.34 g) in cold tetrahydrofuran (10 mL) was added dropwise to a suspension of litium aluminiumhydride (0.34 g) in tetrahydrofuran (20 mL) at 0° C. The mixture was stirred for 15 min and allowed to warn to approx. 10° C., whereafter a solution of the amido compound, prepared above, in tetrahydrofuran (20 mL) was added. The reaction was complete after 1 h and concentrated sodium hydroxide (2 mL) was added, dropwise. Drying agent was added followed by filtration and evaporation to give the crude target base (1.94 g). Addition of oxalic acid (0.49 g) in acetone and filtration gave the title compound as pure white crystalline material (1.77 g). Mp. 106-110° C. (decomposes). 1H NMR (DMSO-d₆): 3.10 (t, 2H); 3.15 (s, 4H); 3.25 (s, 4H); 3.35 (t, 2H); 5.00-6.00 (b, 1H); 6.35 (s, 1H); 6.45 (d, 1H); 7.00 (t, 1H); 7.05 (d, 1H); 7.25-7.35 (m, 2H); 7.50-7.65 (m, 2H). MS m/z: 390 (MH+), 161. Anal. Calcd for C₂₂H₂₁ClFN₃S: C, 53.78; H, 4.71; N, 8.36. Found C, 53.69; H, 4.99; N, 8.51.

The following compounds were prepared analogously:

3b, 4-(4-[2-(2,6-Dichloro-phenylsulfanyl)-ethyl]-piperazin-1-yl)-1H-indole, Oxalate.

Mp. 130-33° C. (decomposes). ¹H NMR (DMSO-d₆): 2.90-3.00 (m, 6H), 3.05-3.20 (s, 4H); 3.20 (t, 2H); 4.40-5.50 (b, 1H); 6.35 (s, 1H); 6.45 (d, 1H); 6.95 (t, 1H); 7.05 (d, 1H); 7.20 (s, 1H); 7.40 (t, 1H); 7.60 (d, 2H). MS m/z: 406 (MH+), 177. Anal. Calcd for C₂₂H₂₁Cl₂N₃S: C, 53.23; H, 4.67; N, 8.46. Found C, 53.12; H, 4.90; N, 8.45.

3c, 4-{4-[2-(3, 4-Dichloro-phenylsulfanyl)-ethyl]-piperazin-1-yl}-1H-indole, 0.8 oxalate.

Mp. 140-41° C. ¹H NMR (DMSO-d₆): 2.90-3.10 (m, 6H); 3.15-3.30 (s, 4H); 3.30-3.40 (t, 2H); 3.60-4.50 (b, 1H); 6.35-6.40 (s, 1H); 6.4576.50 (d, 1H); 6.95-7.00 (t, 1H); 7.05-7.10(d, 1H); 7.25-7.30 (s, 1H); 7.35-7.40 (d, 1H); 7.55-7.60 (d, 1H); 7.15-7.20 (s, 1H). MS m/z: 406 (MH+), 177. Anal. Calcd for C₂₂H₂₁Cl₂N₃S: C, 54.22; H, 4.77; N, 8.78. Found C, 54.01; H, 4.92; N, 8.68.

3d, 4-{4-[2-(4-Fluoro-phenylsulfanyl)-ethyl]-piperazin-l-yl}-1H-indole, 0.9 Oxalate

Mp. 165-67° C. 1H NMR (DMSO-d₆): 2.60-2.70 (m, 6H); 3.10-3.20 (m, 6H); 6.35-6.40 (s, 1H); 6.40-6.50 (d, 1H); 6.90-7.00 (t, 1H); 7.00-7.10 (d, 1H); 7.10-7.25 (m, 3H); 7.40-7.50 (m, 2H). MS m/z: 356 (MH+), 127. Anal. Calcd for C₂₂H₂₁FN₃S: C, 59.97; H, 5.51; N, 9.63. Found C, 559.84; H, 5.58; N, 9.65.

### Example 4

4a, 4-{4-[3-(2-Chloro-4-fluoro-phenylsulfanyl)-propyl/-piperazin-1-yl}-1H-indole.

A solution of 2-chloro-4-fluoro-thiophenol (5.0 g, 30.7 mmol) in tetrahydrofuran (50 mL) was added dropwise at room temperature to a suspension of sodium hydride (38.4 mmol) in ethanol (50 mL) (Caution: generation of hydrogen). The mixture was stirred for additional 30 min after the generation of hydrogen stopped. The solution was then added dropwise (0.3 mL/min) to a solution of 1,3-dibromopropane (159 g, 768 mmol) in ethanol (200 mL) at 60° C. and stirred for 16 h. The mixture was concentrated in vacuo followed by standard work-up (ethyl acetate) giving an oil. Excess 1,3-dibromopropane was removed in in vacuo (60° C., 0.01 mbar) and the oily residue was purified by silicagel flash chromatography (eluent: heptane) to yield 3-(2-chloro-4-fluorophenylthio)-1-bromopropane (5.2 g, 60%) as a colourless oil.

Caesium carbonate (108 mg, 0.33 mmol) was added to a solution of 3-(2-chloro-4-fluorophenylthio)-1-bromopropane (35 mg, 0.12 mmol) and (1H-indole-4-yl)-piperazine (20 mg, 0.10 mmol) in acetonitril (2 mL). The mixture was stirred at 70° C. for 16 h. After 12 h, isocyanomethyl polystyrene (75 mg, 0.08 mmol) was added and the mixture was slowly cooled to room temperature. The resin was filtered and washed with methanol (1×1 mL) and dichloromethane (1×1 mL). The combined liquid phases were concentrated in vacuo to yield a dark brown oil, which was dissolved in ethyl acetate (3 mL) and loaded on a pre-conditioned ion exchange column. The column was washed with methanol (4 mL) and acetonitrile (4 mL), followed by elution of the product using 4 N solution of ammonia in methanol (4.5 mL). After removal of solvents in vacuo, the product was purified by preparative reversed phase HPLC chromatography. The resulting solution was again loaded on a pre-conditioned ion exchange column. As described above, the column was washed with methanol (4 mL) and acetonitrile (4 mL), followed by elution of the product with 4 N solution of ammonia in methanol (4.5 mL). Evaporation of the volatile solvents afforded the title compound as yellow oil (30 mg, 74 μmol, 74%). LC/MS (m/z) 405 (MH+), Rt=6.11, purity 91.0%.

The following compounds where prepared analogously:

4b, 4-{4-[4-(2-Bromo-4-fluoro-phenoxy)-butyl]-piperazin-1-yl}-1H-indole.

LC/MS (m/z) 447 (MH+), Rt=6.20 (method A), purity 98.8%.

4c. 4-{4-[3-(2, 4-Difluoro-phenoxy)-propyl]-piperazin-1-yl})-H-indole.

LC/MS (m/z) 372 (MH+), Rt=2.20 (method A), purity 88.12%.

4d. 4-{4-[4-(2, 6-Dichloro-phenylsulfanyl)-butyl]-piperazin-1-yl}-1H-indole.

LC/MS (m/z) 436 (MH+), Rt=6.53 (method A), purity 80.59%.

4e. 4-{4-[3-(2-Chloro-4-fluoro-phenoxy)-propyl]-piperazin-1-yl}-1H-indole.

LC/MS (m/z) 389 (MH+), Rt=6.11 (method A), purity 97.8%.

4f. 4-{4-[4-(2-Chloro-6-methyl-phenylsulanyl)-butyl-piperazin-1-yl}-1H-indole.

LC/MS (m/z) 415 (MH+), Rt=6.58 (method A), purity 70.2%.

4g. 4-{4-[4-(2,6-Dichloro-4-fluoro-phenoxy)-butyl]-piperazin-1-yl}-1H-indole.

LC/MS (m/z) 437 (MH+), Rt=6.02 (method A), purity 95.1%.

4h. 4-{4-[3-(2-Bromo-4, 6-difluoro-phenoxy)-propyl]-piperazin-1-yl}-1H-indole.

LC/MS (m/z) 451 (MH+), Rt=5.62 (method A), purity 99.5%.

4i. 4-{4-[3-(2, 6-Dichloro-4-fluoro-phenoxy)-propyl]-piperazin-1-yl}-1H-indole.

LC/MS (m/z) 423 (MH+), Rt=6.38 (method A), purity 87.6%.

4j 4-{4-[4-(4-Bromo-2,6-difluoro-phenoxy)-butyl]-piperazin-1-yl}-1H-indole.

LC/MS (m/z) 465 (MH+), Rt=50.74 (method A), purity 95.2%.

4k. 4-{4-[4-(2,6-Dibromo-4-fluoro-phenoxy)-butyl]-piperazin-1-yl}-1H-indole.

LC/MS (m/z) 526 (MH+), Rt=6.18 (method A), purity 100%.

4l. 14-{4-[3-(2, 4, 6-Tribromo-phenoxy)-propyl]-piperazin-1-yl}-1H-indole.

LC/MS (m/z) 573 (MH+), Rt=6.40 (method A), purity 99.6%.

4m. 4-{4-[3-(4-Bromo-2, 6-difluoro-phenoxy)-propyl]-piperazin-1-yl}-1H-indole.

LC/MS (m/z) 451 (MH+), Rt=2.42 (method A), purity 100%.

4n. 1-(3,5-Difluoro-4-{3-[4-(1H-indol-4-yl)-piperazin-1-yl]-propoxy}-phenyl)-propan-1-one.

LC/MS (m/z) 428 (MH+), Rt=5.46 (method A), purity 98.1%.

4o. 3,5-Dibromo-4-{3-[4-(1H-indol-4-yl)-piperazin-1-yl]-propoxy}-benzonitrile.

LC/MS (m/z) 519 (MH+), Rt=5.38 (method A), purity 84.6%.

4p. 4-{4-[2-(2-Bromo-4, 6-difluoro-phenoxy)-ethyl]-piperazin-1-yl}-1H-indole.

LC/MS (m/z) 437 (MH+), Rt=5.35 (method A), purity 74.4%.

4q. 4-{4-[3-(2, 6-Dichloro-phenylsulfanyl)-propyl]-piperazin-1-yl}-1H-indole.

LC/MS (m/z) 421 (MH+), Rt=2.44 (method A), purity 96.7%.

### Example 5

5aa, 4-{4-[2-(2,6-Dimethyl-phenoxy)-ethyl]-piperazin-1-yl}-1H-indole.

To a solution of phenol (1.6 mmol) in DMF (1.6 mL) was added a solution of potassium-tert.-butoxide (1.6 mL, 1.6 mmol, 1.0M in tert.-butanol). The mixture was stirred for 5 min at room temperature. An aliquot of the resulting solution (850 μL) was added to a solution of 2-bromo-1,1-dimethoxyethane (59 mg, 0.35 mmol) in DMF (0.70 mL). The reaction mixture was warmed to 80° C. and stirred for 16 h. After cooling to room temperature, ethyl acetate (6 mL) was added. The organic phase was washed with water (2×4 mL) and dried over sodium sulphate. After evaporation of the volatiles in vacuo, the resulting oil was dissolved in a mixture of dioxane and 3M HCl (4 mL, dioxane: 3M HCl 8:1) and heated to 80° C. for 1 h. After cooling to room temperature, ethyl acetate (6 mL) was added. The organic phase was washed with water (2×4 mL) and dried over sodium sulphate. After evaporation of the volatiles in vacuo, the resulting oil was dissolved in 1,2-dichloroethane (1.80 mL). An aliquot of the resulting solution (600 μL) was added to a solution of 1-[1H-indol-4-yl]piperazine (4.5 mg, 22.4 μmol) in DMF (60 μL), followed by sodium triacetoxyborohydride (30 mg, 0.14, mmol). After shaking the mixture at room temperature for 2 h, a mixture of methanol/water (600 μL, methanol:water 9:1) was added, and the resulting solution was loaded on a pre-conditioned ion exchange column. The column was washed with acetonitrile (2.5 mL) and methanol (2.5 mL), followed by elution of the product with 4 N solution of ammonia in methanol (4.5 mL). After removal of solvents in vacuo, the the title compound was obtained as a colourless oil (5.7 mg, 16.9 μmol, 75%). LC/MS (m/z) 350 (MH+), Rt=2,32 (method B), purity 89,5%.

The following compounds where prepared analogously:

5ab. 4-{4-[4-(2, 6-Dimethyl-phenylsulfanyl)-butyl]-piperazin-1-yl}-1H-indole.

LC/MS (m/z) 394 (MH+), Rt=2.58 (method B), purity 98.14%.

5ac. 4-{4-[2-(2, 4-Dimethyl-phenylsulfanyl)-ethyl]-piperazin-1-yl}-1H-indole.

LC/MS (m/z) 366 (MH+), Rt=2.38 (method A), purity 93.9%.

5ad. 4-{4-[2-(2,3-Dichloro-phenylsulfanyl)-ethyl]-piperazin-1-yl}-1H-indole.

LC/MS (m/z) 406 (MH+), Rt=2.43 (method A), purity 94.09%.

5ae. 4-{4-[2-(2-Allyl-6-chloro-phenoxy)-ethyl]-piperazin-1-yl}1H-indole.

LC/MS (m/z) 396 (MH+), Rt=2.41 (method A), purity 74.45%.

5af. 4-{4-[3-(2-Trifluoromethyl-phenylsulfanyl)-propyl]-piperazin-1-yl}-1H-indole.

LC/MS (m/z) 420 (MH+), Rt=2.48 (method A), purity 80%.

5ag. 4-{4-[3-(3,4-Dichloro-phenylsulfanyl)-propyl]-piperazin-1-yl}-1H-indole.

LC/MS (m/z) 420 (MH+), Rt=2.53 (method A), purity 94.88%.

5ah. 4-{4-[4-(2,4-Dimethyl-phenoxy)-butyl]-piperazin-1-yl}-1H-indole.

LC/MS (m/z) 378 (MH+), Rt=2.47 (method A), purity 76.4%.

5ai. 4-{4-[4-(2-Ethyl-phenoxy)-butyl]-piperazin-1-yl}-1H-indole.

LC/MS (m/z) 378 (MH+), Rt=2.48 (method A), purity 76.62%.

5aj. 4-[4-(4-Phenylsulfanyl-butyl)-piperazin-1-yl]-1H-indole.

LC/MS (m/z) 366 (MH+), Rt=2.05, purity 89.3%.

5ak. 4-{4-[4-(2-Chloro-5-methyl-phenoxy)-butyl]-piperazin-1-yl}-1H-indole.

LC/MS (m/z) 398 (MH+), Rt=2.24 (method B), purity 84.56%.

5al. 4-{4-[2-(2, 5-Dichloro-phenylsulfanyl)-ethyl]-piperazin-1-yl}-1H-indole.

LC/MS (m/z) 406 (MH+), Rt=2.1 (method B), purity 93.74%.

5am. 4-{4-[2-(3-Chloro-phenylsulfanyl)-ethyl]-piperazin-1-yl}-1H-indole.

LC/MS (m/z) 372 (MH+), Rt=2.01 (method B), purity 96.29%.

5an. 4-{4-[2-(2-Chloro-phenylsulfanyl)-ethyl]-piperazin-1-yl}-1H-indole.

LC/MS (m/z) 372 (MH+), Rt=1.93 (method B), purity 96.26%.

5ao. 4-{4-[3-(3-Chloro-phenylsulfanyl)-propyl]-piperazin-1-yl}-1H-indole.

LC/MS (m/z) 386 (MH+), Rt=2.09 (method B), purity 90.84%.

5ap. 3-Chloro-4-(4-[4-1H-indol-4-yl)-piperazin-1-yl]-butoxy)-benzonitrile.

LC/MS (m/z) 409 (MH+), Rt=1.93 (method B), purity 86.56%.

5aq. 4-{4-[4-(3-Chloro-phenylsulfanyl)-butyl]-piperazin-1-yl}-1H-indole.

LC/MS (m/z) 400 (MH+), Rt=2.23 (method B), purity 84.85%.

5ar. 4-{4-[4-(2-Chloro-phenylsulfanyl)-butyl]-piperazin-1-yl}-1H-indole.

LC/MS (m/z) 400 (MH+), Rt=2.14 (method B), purity 84.83%.

5as. 4-{4-[3-(3,4-Dimethyl-phenylsulfanyl)-propyl]-piperazin-1-yl}-1H-indole.

LC/MS (m/z) 380 (MH+), Rt=2.17 (method B), purity 81.48%.

5at. 3-{4-[4-(1H-Indol-4-yl)-piperazin-1-yl]-butoxy}-benzonitrile.

LC/MS (m/z) 375 (MH+), Rt=1.83 (method B), purity 78.43%.

5au. 4-{4-[4-(2,5-Dichloro-phenoxy)-butyl]-piperazin-1-yl}-1H-indole.

LC/MS (m/z) 418 (MH+), Rt=2.23 (method B), purity 79.44%

5av. 4-{4-[4-(3,4-Dimethoxy-phenylsulfanyl)-butyl]-piperazin-1-yl}-1H-indole.

LC/MS (m/z) 426 (MH+), Rt=1.87 (method B), purity 73.1%.

5aw. 4-{4-[3-(4-Trifluoromethyl-phenylsulfanyl)-propyl]-piperazin-1-yl}-1H-indole.

LC/MS (m/z) 420 (MH+), Rt=2.24 (method B), purity 88.9%.

5ax. 4-{4-[3-(4-Trifluoromethoxy-phenylsulfanyl)-propyl]-piperazin-1-yl}-1H-indole.

LC/MS (m/z) 436 (MH+), Rt=2.31 (method B), purity 91.57%.

5ay. 4-{4-[3-(3-Bromo-phenylsulfanyl)-propyl]-piperazin-1-yl}-1H-indole.

LC/MS (m/z) 430 (MH+), Rt=2.15 (method B), purity 91.2%.

5az. 4-{4-[3-(2-Isopropyl-phenylsulfanyl)-propyl]-piperazin-1-yl}-1H-indole.

LC/MS (m/z) 394 (MH+), Rt=2.32 (method B), purity 82.81%.

5ba. 4-{4-[4-(2-Methoxy-phenoxy)-butyl]-piperazin-1-yl}-1H-indole.

LC/MS (m/z) 380 (MH+), Rt=1.79 (method B), purity 93.2%.

5bb. 4-{4-[4-(2-Isopropyl-phenylsulfanyl)-butyl]-piperazin-1-yl}-1H-indole.

LC/MS (m/z) 408 (MH+), Rt=2.4 (method B), purity 85.1%.

Pharmacological Testing

The compounds of the invention were tested in well-recognised and reliable methods. The tests were as follows:

Inhibition of the Binding of ³H-YM-09151-2 to Human Dopamine D₄ Receptors

By this method, the inhibition by drugs of the binding of [³H]YM-09151-2 (0.06 nM) to membranes of human cloned dopamine D_{4.2}-receptors expressed in CHO-cells is determined in vitro. Method modified from NEN Life Science Products, Inc., technical data certificate PC2533-10/96. The results are given in the following Table 1 as IC₅₀-values.

Inhibition of the Binding of [³H]-Spiperone to Human D₃ Receptors

By this method, the inhibition by drugs of the binding [³H]Spiperone (0.3 nM) to membranes of human cloned dopamine D₃-receptors expressed in CHO-cells is determined in vitro. Method modified from R. G. MacKenzie et al. Eur. J. Pharm.-Mol. Pharm. Sec. 1994, 266, 79-85. The results are given in the following Table I as IC₅₀-values.

Inhibition of ³H-5-HT Uptake into Rat Brain Synaptosomes

Using this method, the ability of drugs to inhibit the accumulation of ³H-5-HT into whole rat brain synaptosomes is determined in vitro. The assay was performed as described by Hyttel, J. Psychopharmacology 1978, 60, 13.

The affinity of the compounds of the invention to 5-HT_{1A} receptors was determined by measuring the inhibition of binding of a radioactive ligand at 5-HT_{1A}-receptors as described in the following test:

Inhibition of ³H-5-CT Binding to Human 5-HT_{1A} Receptors.

By this method, the inhibition by drugs of the binding of the 5-HT_{1A}-agonist ³H-5-carboxamido tryptamine (³H-5-CT) to cloned human 5-HT_{1A}-receptors stably expressed in transfected HeLa cells (HA7) (Fargin, A. et al. J. Biol. Chem. 1989, 264, 14848) is determined in vitro. The assay was performed as a modification of the method described by Harrington, M. A. et al. J. Pharmacol. Exp. Ther. 1994, 268, 1098. Human 5-HT_{1A}-receptors (40 μg of cell homogenate) were incubated for 15 minutes at 37° C. in 50 mM Tris buffer at pH 7.7 in the presence of ³H-5-CT. Non-specific binding was determined by including 10 μM of metergoline. The reaction was terminated by rapid filtration through Unifilter GF/B filters on a Tomtec Cell Harvester. Filters were counted in a Packard Top Counter. The results obtained are presented in table 1 below.

TABLE 1

| | Inhibition of | Inhibition of | | Inhibition of | Inhibition of |
|---|---|---|---|---|---|
| | ³H-YM-09151 binding | ³H-5-CT Binding | | ³H-YM-09151 binding | ³H-5-CT Binding |
| Compound | IC₅₀ (nM) or % | IC₅₀ (nM) or % | Compound | IC₅₀ (nM) or % | IC₅₀ (nM) or % |
| No. | inhibition at 100 nM | inhibition at 50 nM | No. | inhibition at 100 nM | inhibition at 50 nM |
| 1 | 92 | 12 | 5ad | 4.1 | 76% |
| 2a | 1.1 | 2.4 | 5ae | 88% | 92% |
| 2b | 1.2 | 2.7 | 5af | 7.2 | 93% |
| 2d | 1.6 | 6.4 | 5ag | 95% | 93% |
| 2e | 2.2 | 4.5 | 5ah | 90% | 86% |
| 3a | 6.6 | 15 | 5ai | 90% | 93% |
| 4a | 0.52 | 9.3 | 5aj | 100% | 77% |
| 4b | 0.66 | 2.4 | 5ak | 93% | 91% |
| 4c | 1.6 | 5.4 | 5al | 96% | 92% |
| 4d | 1.8 | 7.1 | 5am | 83% | 93% |
| 4e | 2.0 | 4.9 | 5an | 83% | 91% |
| 4f | 3.0 | 5.8 | 5ao | 102% | 100% |
| 4g | 4.9 | 2.4 | 5ap | 106% | 100% |
| 4h | 5.4 | 1.4 | 5aq | 98% | 100% |
| 4i | 16 | 1.0 | 5ar | 98% | 104% |
| 4j | 23 | 17 | 5as | 99% | 103% |
| 4k | 26 | 6.7 | 5at | 95% | 92% |
| 4l | 28 | 1.1 | 5au | 97% | 99% |
| 4m | 39 | 1.0 | 5av | 107% | 69% |
| 4n | 230 | 0.72 | 5aw | 99% | 98% |
| 4o | 32 | 0.72 | 5ax | 98% | 81% |
| 4p | 13 | 36 | 5ay | 105% | 96% |
| 4q | 7.2 | 3.5 | 5az | 94% | 98% |
| 5aa | 81% | 78% | 5ba | 80% | 83% |
| 5ab | 95% | 83% | 5bb | 94% | 94% |
| 5ac | 83% | 85% | | | |

The 5-HT_{1A} antagonistic activity of some of the compounds of the invention has been estimated in vitro at cloned 5-HT_{1A}-receptors stably expressed in transfected HeLa hells (HA7). In this test, 5-HT_{1A}-antagonistic activity is estimated by measuring the ability of the compounds to antagonize the 5-HT-induced inhibition of forskolin induced cAMP accumulation. The assay was performed as a modification of the method described by Pauwels, P. J. et al. Biochem. Pharmacol. 1993, 45, 375.

Some of the compounds of the invention have also been tested for their in vivo effect on 5-HT_{1A} receptors in the assay described by Sánchez, C. et al. Eur. J. Pharmacol. 1996, 315, pp 245. In this test, antagonistic effects of test compounds are determined by measuring the ability of the test compounds to inhibit 5-MeO-DMT induced 5-HT syndrome.

Accordingly, as the compounds of the invention show affinities in the described tests, they are considered useful in the treatment of affective disorders, such as depression, generalised anxiety disorder, panic disorder, obsessive compulsive disorders, social phobia, and eating disorders, and neurological disorders such as psychosis.

1. A compound of general formula I 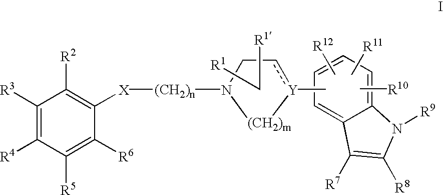
wherein
X is O or S;
n is 2, 3, 4, 5, 6, 7, 8, 9 or 10;
m is 2 or 3;
Y is N, C or CH;
and the dotted line represents an optional bond;
R¹ and R^{1′} independently are hydrogen, or C₁₋₆-alkyl; R⁷, R⁸, R¹⁰, R¹¹ and R¹² are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylsulfanyl, hydroxy, formyl, acyl, amino, C₁₋₆-alkylamino, di(C₁₋₆-alkyl)amino, acylamino, C₁₋₆-alkoxycarbonylamino, aminocarbonylamino, C₁₋₆-alkylaminocarbonylamino and di(₁₋₆-alkyl)aminocarbonylamino;
R⁹ is hydrogen, C₁₋₆-alkyl or acyl;
R², R³, R⁴, R⁵ and R⁶ independently are hydrogen, halogen, cyano, nitro, C₁₋₆-alkyl, C₁₋₆ alkoxy, C₁₋₆-alkylsulfanyl, C₁₋₆ alkylsulfonyl, hydroxy, hydroxy-C₁₋₆-alkyl, C₁₋₆-alkoxycarbonyl, acyl, C₃₋₈-cycloalkyl, C₃₋₈-cycloalkyl-C₁₋₆-alkyl, trifluoromethyl, trifluoromethoxy, NH₂, NR¹³R¹⁴ wherein R¹³ and R¹⁴ independently represent hydrogen, C₁₋₆-alkyl, C₃₋₈-cycloalkyl, or phenyl; or R¹³ and R¹⁴ together with the nitrogen to which they are attached form a 5- or 6-membered carbocyclic ring optionally containing one further heteroatom;
its enantiomers, and a pharmaceutically acceptable acid addition salt thereof.

2. The compound of claim 1 wherein
X is O or S;
n is 2, 3, 4 or 5;
m is 2 or 3;
Y represents N or CH;
R¹ and R^{1′} are both hydrogen;
one or two of R⁷, R⁸, R¹⁰, R¹¹ and R¹² independently are hydrogen, halogen, CF₃, CN or C₁₋₆-alkyl; and the remaining of R⁷, R⁸, R¹⁰, R¹¹ and R¹² are hydrogen;
R⁹ is hydrogen;
R², R³, R⁴, R⁵ and R⁶ independently are hydrogen, halogen, C₁₋₆-alkyl, C₃₋₈-cycloalkyl, C₁₋₆-alkoxy, hydroxy, nitro, CN, CF₃, OCF₃, acyl; NH₂, NR¹³R¹⁴ wherein R¹³ and R¹⁴ independently represent hydrogen, C₁₋₆-alkyl, C₃₋₈-cycloalkyl, or phenyl; or R¹³ and R¹⁴ together with the nitrogen forms a piperidine, morpholine, piperazine or pyrrolidine;
its enantiomers, and a pharmaceutically acceptable acid addition salt thereof.

3. The compound of claim 1 or 2, wherein R¹ and R^{1′} are hydrogen.

4. The compound of claim 1 or 2, wherein m is 2.

5. The compound of claim 1 or 2, wherein n is 2, 3 or 4.

6. The compound of claim 1 or 2, wherein Y is N.

7. The compound of claim 1 or 2, wherein at least one of R², R³, R⁴, R₅ and R⁶ is halogen.

8. The compound of claim 1 or 2, wherein at least two of R², R³, R⁴, R⁵ and R⁶ are halogen.

9. The compound of claim 1 or 2, wherein at least three of R², R³, R⁴, R⁵ and R⁶ are halogen.

10. The compound of claim 1 or 2, wherein R² and/or R⁶ are not hydrogen.

11. The compound of claim 1 or 2, wherein the indole is attached to the group Y in position 4.

12. The compound of claim 1, which is selected from the group consisting of
4-{4-[3-(2-Chloro-phenoxy)-propyl]-piperazin-1-yl}-1H-indole
4-{4-[3-(2-Chloro-phenylsulfanyl)-propyl]-piperazin-1-yl}-1H-indole
4-{4-[3-(2-Bromo-phenylsulfanyl)-propyl]-piperazin-1-yl}-1H-indole
4-{4-[3-(2-Bromo-phenoxy)-propyl]-piperazin-1-yl}-1H-indole
4-{4-[4-(2-Bromo-4-fluoro-phenoxy)-butyl]-piperazin-1-yl}-1H-indole
4-{4-[4-(2-Chloro-6-methyl-phenylsulfanyl)-butyl]-piperazin-1-yl}-1H-indole
4-{4-[2-(2-Chloro-4-fluoro-phenylsulfanyl)-ethyl]-piperazin-1-yl}-1H-indole
4-{4-[2-(2,6-Dichloro-phenylsulfanyl)-ethyl]-piperazin-1-yl}-1H-indole
4-{4-[2-(3,4-Dichloro-phenylsulfanyl)-ethyl]-piperazin-1-yl}-1H-indole
4-{4-[2-(4-Fluoro-phenylsulfanyl)-ethyl]-piperazin-1-yl}-1H-indole
4-{4-[3-(2-Chloro-4-fluoro-phenylsulfanyl)-propyl]-piperazin-1-yl}-1H-indole
4-{4-[4-(2-Bromo-4-fluoro-phenoxy)-butyl]-piperazin-1-yl}-1H-indole
4-{4-[3-(2,4-Difluoro-phenoxy)-propyl]-piperazin-1-yl}-1H-indole
4-{4-[4-(2,6-Dichloro-phenylsulfanyl)-butyl]-piperazin-1-yl}-1H-indole
4-{4-[3-(2-Chloro-4-fluoro-phenoxy)-propyl]-piperazin-1-yl}-1H-indole
4-{4-[4-(2-Chloro-6-methyl-phenylsulfanyl)-butyl]-piperazin-1-yl}-1H-indole
4-{4-[4-(2,6-Dichloro-4-fluoro-phenoxy)-butyl]-piperazin-1-yl}-1H-indole
4-{4-[3-(2-Bromo-4,6-difluoro-phenoxy)-propyl]-piperazin-1-yl}-1H-indole
4-{4-[3-(2,6-Dichloro-4-fluoro-phenoxy)-propyl]-piperazin-1-yl}-1H-indole
4-{4-[4-(4-Bromo-2,6-difluoro-phenoxy)-butyl]-piperazin-1-yl}-1H-indole
4-{4-[4-(2,6-Dibromo-4-fluoro-phenoxy)-butyl]-piperazin-1-yl}-1H-indole
4-{4-[3-(2,4,6-Tribromo-phenoxy)-propyl]-piperazin-1-yl}-1H-indole
4-{4-[3-(4-Bromo-2,6-difluoro-phenoxy)-propyl]-piperazin-1-yl}-1H-indole
1-(3,5-Difluoro-4-{3-[4-(1H-indol-4-yl)-piperazin-1-yl]-propoxy}-phenyl)-propan-1-one
3,5-Dibromo-4-{3-[4-(1H-indol-4-yl)-piperazin-1-yl]-propoxy}-benzonitrile
4-{4-[2-(2-Bromo-4,6-difluoro-phenoxy)-ethyl]-piperazin-1-yl}-1H-indole
4-{4-[3-(2,6-Dichloro-phenylsulfanyl)-propyl]-piperazin-1-yl}-1H-indole
4-{4-[2-(2,6-Dimethyl-phenoxy)-ethyl]-piperazin-1-yl}-1H-indole
4-{4-[4-(2,6-Dimethyl-phenylsulfanyl)-butyl]-piperazin-1-yl}-1H-indole
4-{4-[2-(2,4-Dimethyl-phenylsulfanyl)-ethyl]-piperazin-1-yl}-1H-indole
4-{4-[2-(2,3-Dichloro-phenylsulfanyl)-ethyl]-piperazin-1-yl}-1H-indole
4-{4-[2-(2-Allyl-6-chloro-phenoxy)-ethyl]-piperazin-1-yl}-1H-indole
4-{4-[3-(2-Trifluoromethyl-phenylsulfanyl)-propyl]-piperazin-1-yl}-1H-indole
4-{4-[3-(3,4-Dichloro-phenylsulfanyl)-propyl]-piperazin-1-yl}-1H-indole
4-{4-[4-(2,4-Dimethyl-phenoxy)-butyl]-piperazin-1-yl}-1H-indole
4-{4-[4-(2-Ethyl-phenoxy)-butyl]-piperazin-1-yl}-1H-indole
4-[4-(4-Phenylsulfanyl-butyl)-piperazin-1-yl]-1H-indole
4-{4-[4-(2-Chloro-5-methyl-phenoxy)-butyl]-piperazin-1-yl}-1H-indole
4-{4-[2-(2,5-Dichloro-phenylsulfanyl)-ethyl]-piperazin-1-yl}-1H-indole
4-{4-[2-(3-Chloro-phenylsulfanyl)-ethyl]-piperazin-1-yl}-1H-indole
4-{4-[2-(2-Chloro-phenylsulfanyl)-ethyl]-piperazin-1-yl}-1H-indole
4-{4-[3-(3-Chloro-phenylsulfanyl)-propyl]-piperazin-1-yl}-1H-indole
3-Chloro-4-{4-[4-(1H-indol-4-yl)-piperazin-1-yl]-butoxy}-benzonitrile
4-{4-[4-(3-Chloro-phenylsulfanyl)-butyl]-piperazin-1-yl}-1H-indole
4-{4-[4-(2-Chloro-phenylsulfanyl)-butyl]-piperazin-1-yl}-1H-indole
4-{4-[3-(3,4-Dimethyl-phenylsulfanyl)-propyl]-piperazin-1-yl}-1H-indole
3-{4-[4-(1H-Indol-4-yl)-piperazin-1-yl]-butoxy}-benzonitrile
4-{4-[4-(2,5-Dichloro-phenoxy)-butyl]-piperazin-1-yl}-1H-indole
4-{4-[4-(3,4-Dimethoxy-phenylsulfanyl)-butyl]-piperazin-1-yl}-1H-indole
4-{4-[3-(4-Trifluoromethyl-phenylsulfanyl)-propyl]-piperazin-1-yl}-1H-indole
4-{4-[3-(4-Trifluoromethoxy-phenylsulfanyl)-propyl]-piperazin-1-yl}-1H-indole
4-{4-[3-(3-Bromo-phenylsulfanyl)-propyl]-piperazin-1-yl}-1H-indole
4-{4-[3-(2-Isopropyl-phenylsulfanyl)-propyl]-piperazin-1-yl}-1H-indole
4-{4-[4-(2-Methoxy-phenoxy)-butyl]-piperazin-1-yl}-1H-indole or
4-{4-[4-(2-Isopropyl-phenylsulfanyl)-butyl]-piperazin-1-yl}-1H-indole or a pharmaceutically acceptable salt thereof.

13. A pharmaceutical composition comprising at least one compound of claim 1 according to any of the preceding claims or a pharmaceutically acceptable acid addition salt thereof or prodrug thereof in a therapeutically effective amount and in combination with one or more pharmaceutically acceptable carriers or diluents.

14. A method for the treatment of diseases or disorders in humans responsive to ligands of the 5-HT₁ₐ-receptor potentially in combination with serotonine reuptake and/or ligands at the dopamine D₄-receptor, comprising administering an effective amount of a compound of claim 1.

15. A method according to claim 14 wherein said diseases or disorders are selected from the group consisting of affective disorders, eating disorders, and neurological disorders.

16. A method according to claim 15, wherein said diseases or disorders are affective disorders selected from the group consisting of generalised anxiety disorder, panic disorder, obsessive compulsive disorder, depression, and social phobia.
